(19) United States
(12) Patent Application Publication
(10) Pub. No.: US 2026/0260353 A1
(43) Pub. Date: Sep. 3, 2026

(54) INFORMATION PROCESSING APPARATUS, METHOD, AND SYSTEM

(71) Applicant: CaTe Inc., Tokyo (JP)

(72) Inventor: Kazuhiro TERASHIMA, Bunkyo-ku (JP)

(73) Assignee: CaTe Inc., Tokyo (JP)

(21) Appl. No.: 19/655,005

(22) Filed: Apr. 22, 2026

Related U.S. Application Data
(63) Continuation of application No. PCT/JP2024/024530, filed on Jul. 8, 2024.

(30) Foreign Application Priority Data
Oct. 27, 2023 (JP) 2023-184816

(51) Int. Cl. G06T 7/00; A61B 5/00; A61B 5/11; G06T 7/246; G06V 10/70; G06V 40/16; G06V 40/18; G06V 40/20; G10L 25/66; G16H 50/20

(52) U.S. Cl. CPC G06T 7/0014; A61B 5/11; A61B 5/4803; A61B 5/7267; G06T 7/248; G06V 10/70; G06V 40/174; G06V 40/18; G06V 40/23; G10L 25/66; G16H 50/20; G06T 2207/10016; G06T 2207/20081; G06T 2207/30201; G06V 2201/03

(57) ABSTRACT

An information processing apparatus includes processing circuitry. The processing circuitry is configured to: acquire type information on a first type that is an exercise type selected from a plurality of exercise types by a user, a related party, or an algorithm; acquire user movement information on the user's movement resulting from analyzing an image in which the user is captured performing exercise of the first type; and evaluate the user for a sign of a target disease on the basis of at least the user movement information and a feature related to a symptom of the target disease, the target disease being at least one of a cranial nerve disease or a peripheral nerve/muscle disease, associated with the first type.

### CROSS-REFERENCE TO RELATED APPLICATIONS

This application is based upon and claims the benefit of priority from the prior Japanese Patent Application No. 2023-184816, filed Oct. 27, 2023 and from PCT Patent Application No. PCT/JP2024/24530, filed Jul. 8, 2024, the entire contents of which are incorporated herein by reference.

### FIELD

The present disclosure relates to an information processing apparatus, a method, and a system.

### BACKGROUND

The progression of cranial nerve diseases such as dementia may be slowed or their symptoms alleviated through early detection and prompt treatment initiation. While symptoms of cranial nerve diseases vary, such a disease in an exemplary case may affect a patient's gait, leading to a gait disorder.

The conventional literature discloses a technical concept for the automatic diagnosis of neurological diseases such as Parkinson's disease. This technical concept intends to focus on posture, tremor, and gait in a patient's physical movements so that a motor disorder appearing in at least one of these factors is measured in a simple and quantitative manner.

The technical concept disclosed in the conventional literature requires a patient to have more than a dozen acceleration sensors attached to the patient's body for posture measurement. Thus, adopting this technical concept to routinely measure the patient's motor disorder imposes a significant burden on the patient and is hardly considered practical.

An object of the present disclosure is to provide a technique for evaluating a user for signs of abnormalities while minimizing the burden on the user.

### BRIEF DESCRIPTION OF THE DRAWINGS

FIG. 1 is a block diagram illustrating the configuration of an information processing system in an embodiment.

FIG. 2 is a block diagram illustrating the configuration of a client device in the embodiment.

FIG. 3 is a block diagram illustrating the configuration of a server in the embodiment.

FIG. 4 is an explanatory diagram of an implementation of the embodiment.

FIG. 5 is a diagram illustrating the data structure of an exercise type database in the embodiment.

FIG. 6 is a diagram illustrating the data structure of a model database in the embodiment.

FIG. 7 is a flowchart of a sign evaluation process in the embodiment.

FIG. 8 is a diagram illustrating an exemplary screen displayed in the sign evaluation process in the embodiment.

FIG. 9 is a diagram illustrating an exemplary screen displayed in the sign evaluation process in the embodiment.

FIG. 10 is a diagram illustrating an exemplary screen displayed in the sign evaluation process in the embodiment.

FIG. 11 is a diagram illustrating the data structure of a pattern database in Variation 1.

### DETAILED DESCRIPTION

In general, according to an embodiment, an information processing apparatus includes processing circuitry. The processing circuitry is configured to: acquire type information on a first type that is an exercise type selected from a plurality of exercise types by a user, a related party, or an algorithm; acquire user movement information on the user's movement resulting from analyzing an image in which the user is captured performing exercise of the first type; and evaluate the user for a sign of a target disease on the basis of at least the user movement information and a feature related to a symptom of the target disease, the target disease being at least one of a cranial nerve disease or a peripheral nerve/muscle disease, associated with the first type.

An embodiment of the present invention will be described in detail below with reference to the drawings. Note that like components in the drawings for describing the embodiment are basically given like reference symbols and will not be described repeatedly.

### (1) Configuration of an Information Processing System

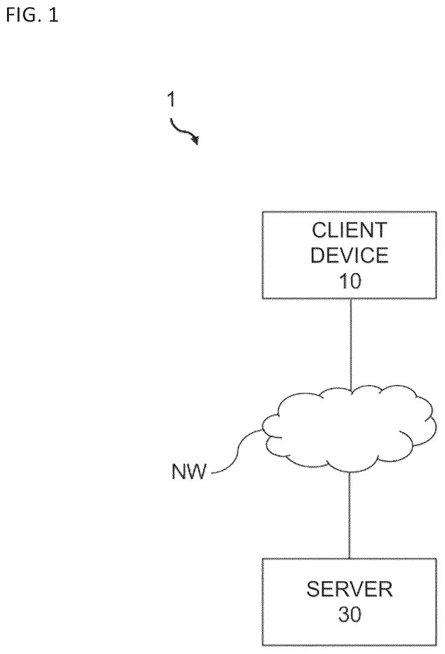

The configuration of an information processing system will be described. FIG. 1 is a block diagram illustrating the configuration of the information processing system in the present embodiment.

As shown in FIG. 1, an information processing system 1 includes a client device 10 and a server 30.

The client device 10 may vary in number, for example, depending on the number of users. As such, there may be two or more client devices 10. The information processing system 1 may further include a terminal of a related party for each user.

The related party may include, for example, at least one of the following:
the user's family
a person who planned/plans exercise therapy for the user (typically, a healthcare worker (e.g., a physician (which may include the user's attending physician), a nurse, a pharmacist, a physical therapist, an occupational therapist, or a clinical laboratory technologist) or a dietitian)
a person who instructs exercise therapy for the user (typically, a healthcare worker, a dietitian, or a trainer)

The client device 10 and the server 30 are interconnected via a network (e.g., the Internet or an intranet) NW.

The client device 10 is an example of an information processing apparatus that transmits requests to the server 30. The client device 10 is, for example, a smartphone, a tablet terminal, or a personal computer.

The server 30 is an example of an information processing apparatus that provides, to the client device 10, responses to requests transmitted from the client device 10. The server 30 is, for example, a server computer.

### (1-1) Configuration of the Client Device

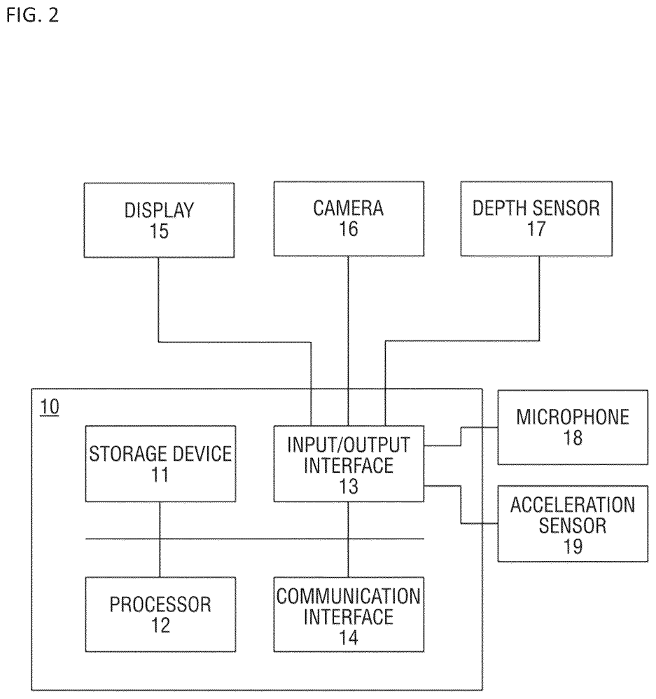

The configuration of the client device will be described. FIG. 2 is a block diagram illustrating the configuration of the client device in the present embodiment.

As shown in FIG. 2, the client device 10 includes a storage device 11, a processor 12, an input/output interface 13, and a communication interface 14. The client device 10 is connected to a display 15, a camera 16, a depth sensor 17, a microphone 18, and an acceleration sensor 19.

The storage device 11 is configured to store programs and data. The storage device 11 is, for example, a combination of read only memory (ROM), random access memory (RAM), and storage (e.g., flash memory or a hard disk).

The programs include, for example, the following programs:
operating system (OS) programs
the programs of applications that perform information processing (e.g., a web browser, a therapeutic app, a rehabilitation app, or a fitness app)

The therapeutic app or the rehabilitation app is intended for diseases for which exercise may contribute to symptom alleviation, for example, heart diseases, lifestyle-related diseases (hypertension, diabetes, dyslipidemia, and hyperlipidemia), and obesity.

The data includes, for example, the following data:
databases referred to during information processing
data resulting from performing information processing, (i.e., the results of performing information processing)

The processor 12 is a computer that implements the functions of the client device 10 by running programs stored in the storage device 11. The processor 12 is, for example, at least one of the following:
a central processing unit (CPU)
a graphics processing unit (GPU)
an application specific integrated circuit (ASIC)
a field programmable gate array (FPGA)

The input/output interface 13 is configured to receive information (e.g., user instructions, images, and sound) from input devices connected to the client device 10 and to output information (e.g., images and commands) to output devices connected to the client device 10.

The input devices include, for example, the camera 16, the depth sensor 17, the microphone 18, the acceleration sensor 19, a keyboard, a pointing device, a touch panel, a sensor, or a combination thereof.

The output devices may include, for example, the display 15, a speaker, or a combination thereof.

The communication interface 14 is configured to control communication between the client device 10 and external devices (e.g., another client device 10 such as the related party's terminal, and the server 30).

Specifically, the communication interface 14 may include a module for communication (e.g., a Wi-Fi module, a mobile communication module, a Bluetooth (R) module, or a combination thereof).

The display 15 is configured to display images (still images or video). The display 15 is, for example, a liquid crystal display or an organic EL display.

The camera 16 is configured to capture images and generate image signals.

The depth sensor 17 is, for example, a light detection and ranging (LIDAR) sensor. The depth sensor 17 is configured to measure the distance (depth) from the depth sensor 17 to an object (e.g., the user) in the surroundings. Note that the depth sensor 17 is not essential and may be eliminated from the client device 10.

The microphone 18 is configured to receive sound waves and generate sound signals. The microphone 18 is preferably disposed close to the user's body (in particular, respiratory system), as in the case of an earpiece microphone.

The acceleration sensor 19 is configured to detect acceleration.

### (1-2) Configuration of the Server

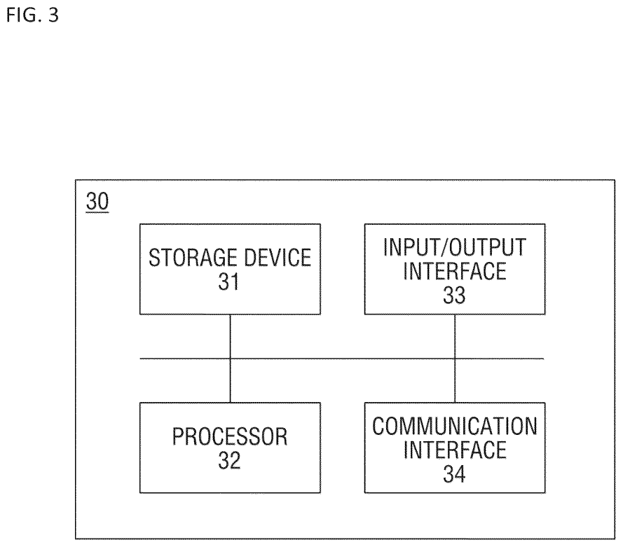

The configuration of the server will be described. FIG. 3 is a block diagram illustrating the configuration of the server in the present embodiment.

As shown in FIG. 3, the server 30 includes a storage device 31, a processor 32, an input/output interface 33, and a communication interface 34.

The storage device 31 is configured to store programs and data. The storage device 31 is, for example, a combination of ROM, RAM, and storage.

The programs include, for example, the following programs:
OS programs
the programs of applications that perform information processing

The data includes, for example, the following data:
databases referred to during information processing
the results of performing information processing

The processor 32 is a computer that implements the functions of the server 30 by running programs stored in the storage device 31. The processor 32 is, for example, at least one of the following:
a CPU
a GPU
an ASIC
an FPGA

The input/output interface 33 is configured to receive information (e.g., user instructions) from input devices connected to the server 30 and to output information to output devices connected to the server 30.

The input devices include, for example, a keyboard, a pointing device, a touch panel, or a combination thereof.

The output devices include, for example, a display.

The communication interface 34 is configured to control communication between the server 30 and external devices (e.g., client devices 10).

### (2) An Implementation of the Embodiment

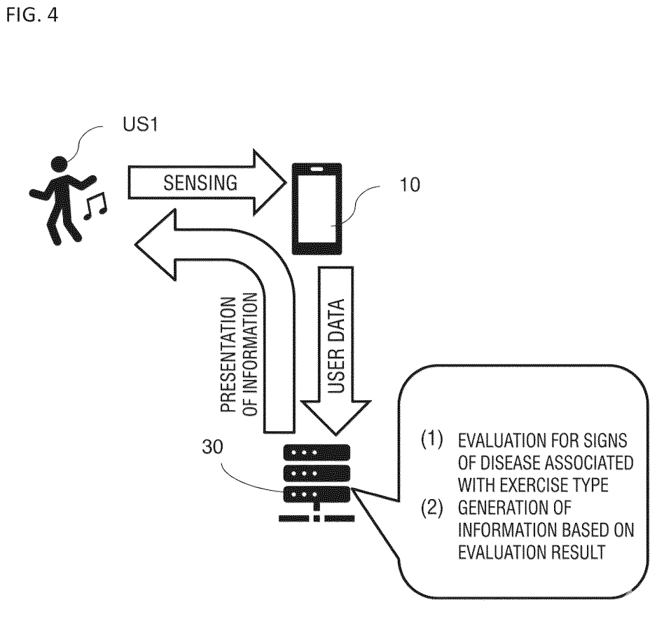

An implementation of the present embodiment will be described. FIG. 4 is an explanatory diagram of an implementation of the present embodiment.

As shown in FIG. 4, the client device 10 senses a user US1 performing exercise. Note that although not shown, the user US1 performing exercise may further be sensed by, for example, a wearable device having an acceleration sensor. The user US1 is typically a person receiving exercise therapy, for example, but not limited to, a participant in a (cardiac) rehabilitation program or an exercise instruction program.

The user US1 is performing a type of exercise (hereinafter referred to as a “first type”) selected from multiple available exercise types by the user US1, the related party, or an algorithm. The available exercise types are associated with a certain disease in the storage device 31 of the server 30 or in any other storage device accessible by the server 30.

The disease associated with the exercise types may be a cranial nerve disease, a peripheral nerve/muscle disease, or a combination thereof. The cranial nerve disease refers specifically to a cranial nerve disease primarily involving an intracranial disease. Examples of the cranial nerve disease may include at least one of dementia (e.g., Alzheimer's, vascular, or Lewy body), normal pressure hydrocephalus, or a cerebellar disease. Examples of the peripheral nerve/muscle disease may include at least one of Parkinson's disease, myositis, muscular dystrophy, or Charcot-Marie-Tooth disease.

As an example, the camera 16 captures the appearance (e.g., whole body) of the user US1 performing exercise, for example from a distance of approximately two meters, either from the front or from a diagonal front angle. The camera 16 may be disposed at an appropriate height using a tripod or any other height adjustment tool. The depth sensor 17 measures the distance (depth) from the depth sensor 17 to each part of the user US1. Note that three-dimensional video data may be generated by combining two-dimensional video data generated by, for example, the camera 16 and depth data generated by, for example, the depth sensor 17. The microphone 18 receives sound emitted by the user US1 performing exercise (e.g., sound produced by breathing or vocalization) and generates sound signals. The acceleration sensor 19 measures acceleration during the exercise of the user US1.

The client device 10 acquires various sorts of sensing data, and analyzes the data as necessary. As an example, the client device 10 may refer to video data acquired from the camera 16 to analyze the body movements of the user US1 performing exercise (in particular, the movements of the skeleton or other feature points over multiple timepoints, or the state of the skeleton or other feature points at a single timepoint). The client device 10 may further refer to depth data acquired from the depth sensor 17 to analyze the body movements of the user US1 performing exercise. The client device 10 transmits, to the server 30, user data that includes at least one of the sensing data or the result of analyzing the sensing data. However, some or all of the analysis may be performed by the server 30.

On the basis of the user data acquired from the client device 10, the server 30 evaluates the user US1 for at least one of a cranial nerve disease or a peripheral nerve/muscle disease (hereinafter referred to as a “target disease”) associated with the first type. Specifically, the server 30 performs the evaluation on the basis of a feature related to a symptom (e.g., a specific type of motor disorder) of the target disease, and on the basis of the result of analyzing the user's body movements.

The server 30 generates information that is based on the evaluation result and presents the information to the user US1 via the client device 10. Alternatively, the server 30 may present the information to the above-described related party instead of the user US1. Thus, in a case where the evaluation result indicates a sign of the target disease in the user US1, the user US1 can be prompted to, for example, consult a medical institution or take a test. In addition, the present embodiment does not require the user US1 to wear numerous sensors (e.g., attach accelerometers to all over the body) or to perform only a limited type of exercise (e.g., walking only). This reduces the burden on the user US1 and thus facilitates routinely performing the evaluation (e.g., as part of exercise therapy), contributing to early detection of the target disease.

### (3) Databases

Databases in the present embodiment will be described. The following databases are stored in the storage device 31 or in any other storage device accessible by the server 30.

### (3-1) Exercise Type Database

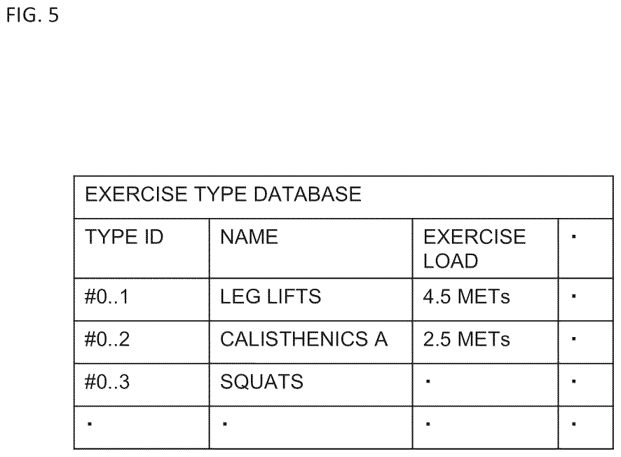

An exercise type database in the present embodiment will be described. FIG. 5 is a diagram illustrating the data structure of the exercise type database in the present embodiment.

The exercise type database stores exercise type information. The exercise type information is information on exercise types (e.g., the above-described available exercise types). The exercise types in the present embodiment include those that can be performed without using equipment capable of adjusting the exercise load, for example, calisthenics, bodyweight training, dance, walking, running, and treadmill exercise. These exercise types offer a wide variety, including those performed in a standing position. Furthermore, the exercise loads of these exercise types in the present embodiment are adjustable through form (e.g., the range of movement of a body part, or the degree of arm or leg spread), pace, the number of reps, or the duration or frequency of breaks. However, the exercise types in the present embodiment may further include those performed using equipment capable of adjusting the exercise load, for example, strength training using an ergometer or a training gear.

As shown in FIG. 5, the exercise type database includes a “type ID” field, a “name” field, and an “exercise load” field. The fields are associated with each other.

The “type ID” field stores exercise type IDs. Each exercise type ID is information that identifies the exercise type corresponding to the relevant record.

The “name” field stores exercise type name information. Each item of the exercise type name information is information on the name of the exercise type corresponding to the relevant record.

The “exercise load” field stores exercise load information. Each item of the exercise load information is information on the standard exercise load of the exercise type corresponding to the relevant record. The standard load refers to, for example, information on the exercise load of the corresponding exercise type performed by a person with standard physical capabilities. As an example, this exercise load may be derived by actually measuring exercise loads on people performing the corresponding exercise type and statistically processing (e.g., averaging) the measurement results, or may be obtained by referring to an exercise load that is set by a third-party entity for the corresponding exercise type. The exercise load information may be managed at a finer granularity than the exercise types. As an example, for each exercise type, different exercise load information items may be managed for variations that are different in form, pace, the number of reps, or the duration or frequency of breaks. Note that the “exercise load” field is not essential and may be eliminated from the exercise type database.

### (3-2) Model Database

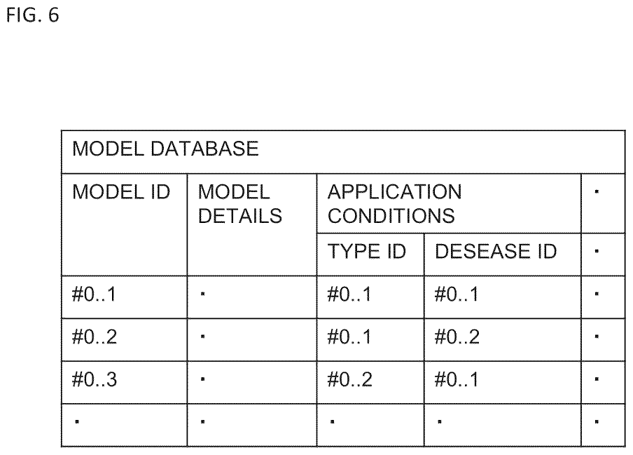

A model database in the present embodiment will be described. FIG. 6 is a diagram illustrating the data structure of the model database in the present embodiment.

The model database stores model information. The model information is information on trained models that perform inference regarding signs of one or more diseases on the basis of input data on at least body movements (e.g., the skeleton).

As shown in FIG. 6, the model database includes a “model ID” field, a “model details” field, and an “application conditions” field. The fields are associated with each other.

The “model ID” field stores model IDs. Each model ID is information that identifies the trained model corresponding to the relevant record.

The “model details” field stores model details information. Each item of the model details information is information on the details of the trained model corresponding to the relevant record. The details information includes information capable of identifying the structure of the trained model (e.g., the layer structure, the connection relationships between nodes, and the weight of each edge, in a neural network). For example, the details information may include information indicating the values of parameters defining the structure of the trained model, or information indicating the location where such values are stored.

The “application conditions” field stores application condition information. Each item of the application condition information is information on a condition under which the trained model corresponding to the relevant record is applicable. As an example, the application condition information may include information capable of identifying an exercise type (e.g., the above-described type ID) and information capable of identifying a disease (i.e., a target disease). As the information capable of identifying a disease, a disease ID may be defined that uniquely identifies a single disease, or a disease group including multiple diseases.

### (3-3) Other Databases

Databases other than those described above may also be constructed. As an example, a user profile database will be described.

The user profile database stores user profile information. The user profile information is information on the profiles of the users of the information processing system 1 (e.g., people receiving exercise therapy).

The user profile database may store records, each including at least one of the following information items:
a user ID
user name information
physical information
information indicating the appropriate exercise load for the user
information indicating the related party for the user

The user ID is information that identifies the user corresponding to the relevant record.

The user name information is information on the name (e.g., full name or account name) of the user corresponding to the relevant record.

The physical information is information on the user's body (capabilities) corresponding to the relevant record. As an example, the physical information may include information on the user's age, sex, weight, height, and diseases.

### (4) Information Processing

Information processing in the present embodiment will be described.

### (4-1) A Sign Evaluation Process

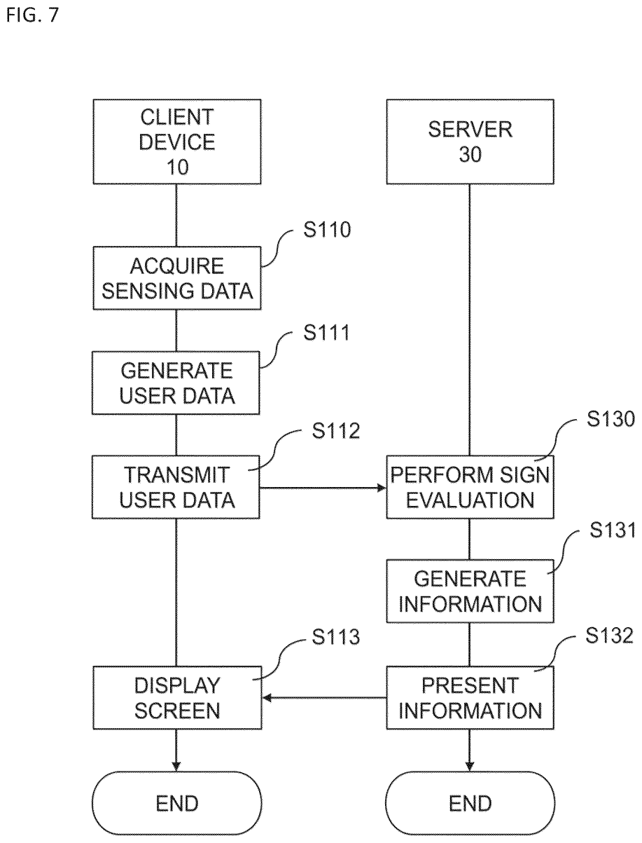

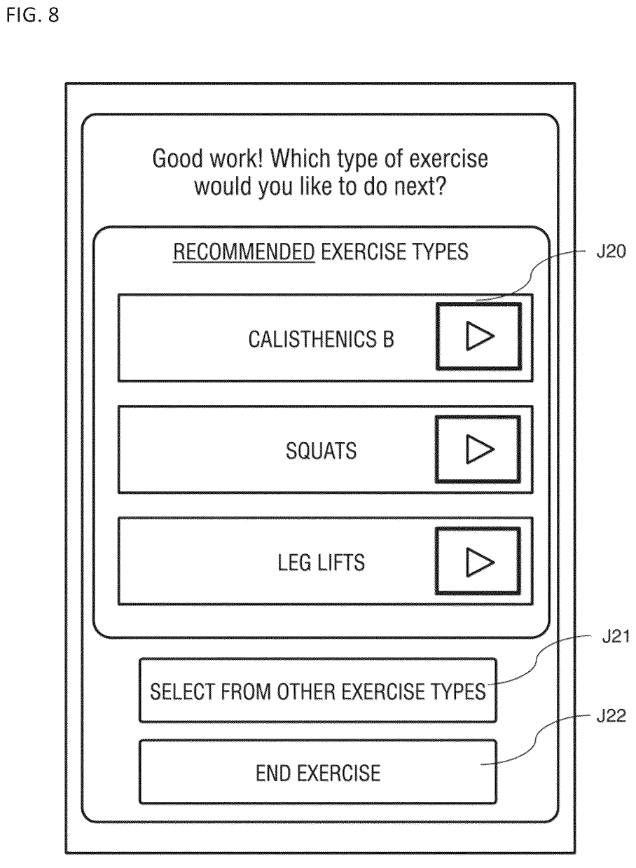

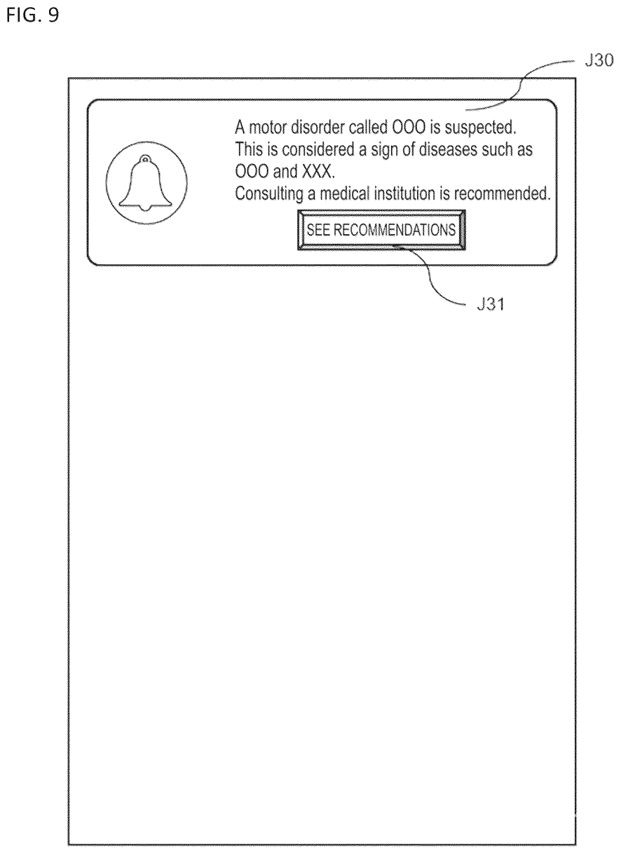

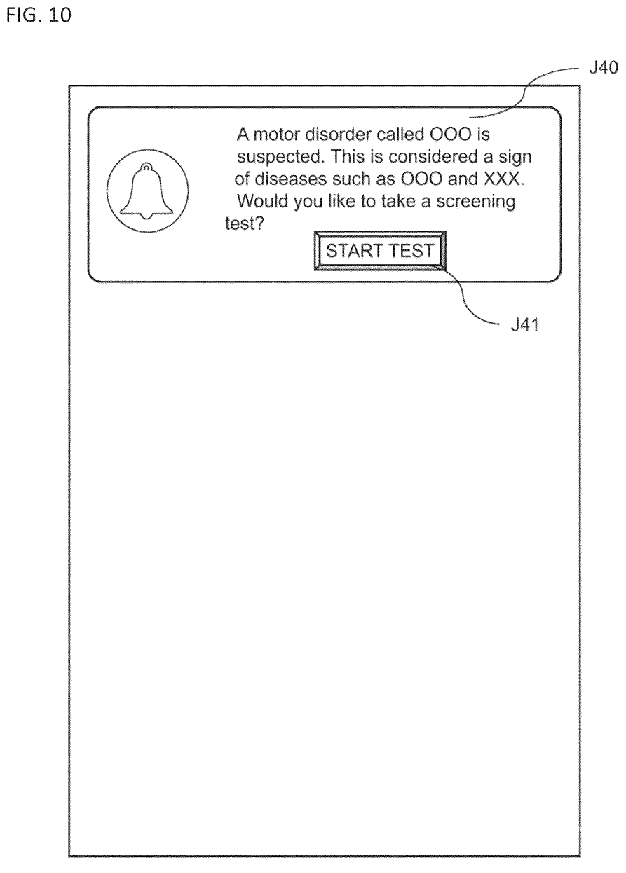

A sign evaluation process in the present embodiment will be described. FIG. 7 is a flowchart of the sign evaluation process in the present embodiment. FIG. 8 is a diagram illustrating an exemplary screen displayed in the sign evaluation process in the present embodiment. FIG. 9 is a diagram illustrating an exemplary screen displayed in the sign evaluation process in the present embodiment. FIG. 10 is a diagram illustrating an exemplary screen displayed in the sign evaluation process in the present embodiment.

The sign evaluation process starts in response to, for example, the fulfillment of any of the following start conditions:
The sign evaluation process is invoked by another process.
The user or the related party for the user performs an operation for invoking the sign evaluation process.
The client device 10 enters a predetermined state (e.g., a predetermined application is launched).
A predetermined date and time arrives.
A predetermined time period elapses since a predetermined event.

As shown in FIG. 7, the client device 10 acquires sensing data (S110).

Specifically, the client device 10 may start capturing a video of the user performing exercise (hereinafter referred to as a “user video”) by enabling the operation of the camera 16. The client device 10 may start measuring the distance from the depth sensor 17 to each part of the user performing exercise (hereinafter referred to as “user depth”) by enabling the operation of the depth sensor 17. The client device 10 may start collecting sound (e.g., sound produced by the user's vocalization or breathing, hereinafter referred to as “user sound”) by enabling the operation of the microphone 18. The client device 10 may start measuring acceleration by enabling the operation of the acceleration sensor 19.

The client device 10 then acquires sensing data from each sensor.

Specifically, the client device 10 acquires sensing results generated by the sensors enabled at step S110. For example, the client device 10 may acquire user video data from the camera 16, user depth data from the depth sensor 17, user sound data from the microphone 18, and user acceleration data on the user's acceleration (hereinafter referred to as “user acceleration”) from the acceleration sensor 19.

After step S110, the client device 10 generates user data (S111).

Specifically, the client device 10 generates user data on the basis of the sensing data acquired at step S110. The user data may include at least one of the following:
data acquired at step S111 (e.g., the user video data, user depth data, user sound data, or user acceleration data)
data obtained by processing the data acquired at step S111
data obtained by analyzing the data acquired at step S111 (e.g., movement information (e.g., skeleton data), facial expression data, gaze data, tremor data, assistive device data, or voice data to be described below, or a combination thereof)
information capable of identifying the exercise type (first type) performed by the user at step S110

After step S111, the client device 10 transmits the user data (S112).

Specifically, the client device 10 transmits the user data generated at step S111 to the server 30.

After step S112, the server 30 performs sign evaluation (S130).

Specifically, the server 30 receives the user data transmitted by the client device 10 at step S112. The server 30 acquires user skeleton information on the basis of the user data acquired from the client device 10. The user skeleton information is information on the user's skeleton, obtained by analyzing the user video data. In addition to the user video data, the user depth data may be referred to for generating the user skeleton information. The user skeleton information may be included in the user data or may be generated by the server 30 analyzing data included in the user data. Note that the user skeleton information is an example of information on the body movements of the user performing exercise (hereinafter referred to as “user movement information”). The user skeleton information may be used along with or replaced by information on the movements of other feature points.

The user skeleton information is information on the skeleton of the user performing exercise (e.g., data such as feature values). The user skeleton information may include, for example, information on the position, velocity, or acceleration of each body part of the user (which may include information on changes in muscle parts used by the user or information on the user's core instability). The user skeleton information can be obtained by referring to the user video data (or the user video data and the user depth data) to analyze the skeleton of the user performing exercise. As an example, the skeleton can be analyzed using Vision, which is an SDK for iOS (R) 14, or other skeleton detection algorithms (e.g., OpenPose, PoseNet, and MediaPipe Pose).

Note that the result of the movement detection such as the skeleton detection can also be used for quantitative or qualitative evaluation of exercise, or a combination thereof. As a first example, the result of the movement detection can be used to count the number of reps. As a second example, the result of the movement detection can be used to evaluate the user's form during the exercise or the appropriateness of the load imposed by the exercise. For example, assume that the exercise type is squats. The result of the movement detection can be used to evaluate whether the knees are not sticking out excessively to create a dangerous form, or whether the hips are being lowered deep enough for a sufficient load.

The server 30 also acquires information on the first type (e.g., information capable of identifying the first type, such as the type ID corresponding to the first type). This information may be included in the user data. Alternatively, this information may be transmitted from the related party's terminal to the server 30 or generated by the server 30 during the process in which the related party or an algorithm selects the first type.

The server 30 evaluates the user for signs of a target disease on the basis of at least the user movement information and features related to symptoms of the target disease. The target disease is at least one of a cranial nerve disease or a peripheral nerve/muscle disease, associated with the first type.

As a first example of the sign evaluation (S130), the server 30 refers to the model database (FIG. 6) to extract records that include, as an application condition, the value of the type ID corresponding to the first type. In this case, the trained model corresponding to each record extracted constitutes a feature related to a symptom of a target disease. The server 30 evaluates the user for a sign of the target disease by applying the trained model to input data that is based on the user movement information.

The trained model focuses on the presence or absence of a characteristic posture or movement of the skeleton or other feature points observed as a symptom of the corresponding disease, thereby inferring the presence or absence of a sign of the symptom, or the degree of possibility of a sign of the symptom. The trained model receives input data that is based on the movement information (e.g., skeleton information) and outputs an evaluation result. The evaluation result may be binary data representing the presence or absence of a sign of any disease, or may be multi-valued data representing the possibility of the sign on a multi-level scale. The trained model may be a trained model created through supervised learning on a training dataset, or a derivative model or distilled model of such a trained model.

A second example of the sign evaluation (S130) is a case where the target disease is specified. The server 30 refers to the model database (FIG. 6) to extract records that include, as application conditions, the values of the type ID corresponding to the first type and the disease ID corresponding to the specified target disease. In this case, the trained model corresponding to each record extracted constitutes a feature related to a symptom of the target disease. The server 30 evaluates the user for a sign of the target disease by applying the trained model to input data that is based on the user movement information. The target disease may be specified by the user, the related party for the user, or an algorithm. Information capable of identifying the specified target disease may be included in the user data, may be transmitted to the server 30 from the related party's terminal, or may be generated by the server 30.

After step S130, the server 30 generates information (S131).

Specifically, on the basis of the result of the evaluation at step S130, the server 30 generates information to be presented to the user.

As a first example of the information generation (S131), assume that the evaluation result indicates a sign of any target disease, or the possibility of a sign of any target disease exceeding a threshold (an example of “the result of evaluating the user satisfies a predetermined condition”). The server 30 may select exercise types to be recommended to the user to perform next (hereinafter referred to as “second types”) and generate information capable of identifying the selected second types (e.g., type IDs). As an example, the server 30 may select, as the second types, types different from the first type from among those associated with the target disease for which the evaluation result at step S131 indicates a sign or indicates the possibility of the sign exceeding a threshold. The server 30 may further select the second types in view of the appropriate exercise load for the user and the exercise load of each exercise type. Re-sensing the user performing such a second type recommended in this manner and re-evaluating the user for signs (S130) can provide a more reliable evaluation result. That is, this enables determining whether the sign observed during the performance of the first type is merely accidental or is observed regardless of exercise type.

As a second example of the information generation (S131), assume that the evaluation result indicates a sign of any target disease, or the possibility of a sign of any target disease exceeding a threshold. The server 30 may generate information capable of identifying medical institutions associated with that target disease. For example, information on medical institutions suitable for diagnosing each target disease may be associated with the target disease and stored in a database in advance. The server 30 can refer to this database to generate the information. The server 30 may narrow down the medical institutions in view of information on the user's place of residence or current location, in addition to the target disease.

As a third example of the information generation (S131), assume that the evaluation result indicates a sign of any target disease, or the possibility of a sign of any target disease exceeding a threshold. The server 30 may generate information recommending the user to take a test associated with that target disease (e.g., a screening test such as Hasegawa's dementia scale). For example, information on a test suitable for each target disease may be associated with the target disease and stored in a database in advance. The server 30 can refer to this database to generate the information.

As a fourth example of the information generation (S131), assume that the evaluation result indicates a sign of any target disease, or the possibility of a sign of any target disease exceeding a threshold. The server 30 may combine two or more of the above first to third examples. For example, the server 30 may perform the above first example at least once and, in a case where the total number or percentage of evaluation results indicating a sign of any target disease or the possibility of the sign exceeding a threshold reaches a predetermined value, the server 30 may perform at least one of the above second or third example.

Note that, for an evaluation result indicating no signs of any target disease or no possibility of signs of any target disease exceeding a threshold, the server 30 may skip the steps of generating information (S131) and presenting the information (S132).

After step S131, the server 30 presents the information (S132).

Specifically, the server 30 transmits the information generated at step S131 to the client device 10. Note that the server 30 may present the information to the related party for the user, in addition to or instead of the user. In that case, the server 30 may transmit the information generated at step S132 to the related party's terminal.

After step S132, the client device 10 displays a screen (S113).

Specifically, the client device 10 receives the information transmitted by the server at step S131. The client device 10 displays, on the display 21, a screen that is based on the received information; the screen is not limited to an application screen and may include a notification (an application notification, or a message such as an email or chat message).

As a first example of the screen display (S113), the client device 10 displays a screen in FIG. 8 on the display 21. The screen in FIG. 8 includes objects J20 to J22.

The objects J20 show information on the above-described second types. The objects J20 also receive user instructions for starting the corresponding exercise types or playing demonstration videos of the corresponding exercise types. In response to the selection of an object J20, the client device 10 may either repeat the sign evaluation process in the present embodiment so that the exercise type (second type) corresponding to the selected object J20 serves as a new first type, or play a demonstration video of the corresponding exercise type.

The object J21 receives a user instruction for selecting an exercise type from among those other than the second types. In response to the selection of the object J21, the client device 10 may, for example, display a list of exercise types other than the second types and receive a user instruction for selecting an exercise type from the list. Upon receiving the user instruction, the client device 10 may play a demonstration video of the exercise type selected by the user. Alternatively, in a case where the selected exercise type is associated with the target disease, the client device 10 may repeat the sign evaluation process in the present embodiment so that the selected exercise type serves as a new first type.

The object J22 receives a user instruction for terminating the exercise. In response to the selection of the object J22, the client device 10 terminates the sign evaluation process in the present embodiment.

As a second example of the screen display (S113), the client device 10 displays a screen in FIG. 9 on the display 21. The screen in FIG. 9 includes objects J30 and J31.

The object J30 shows information that recommends consulting a medical institution.

The object J31 receives a user instruction for viewing information on medical institutions associated with the target disease. Upon receiving the user instruction, the client device 10 may place, on the screen, objects showing information on medical institutions associated with the target disease, or may transition to a screen showing such information.

As a third example of the screen display (S113), the client device 10 displays a screen in FIG. 10 on the display 21. The screen in FIG. 10 includes objects J40 and J41.

The object J40 shows information that recommends taking a test associated with the target disease.

The object J41 receives a user instruction for starting the test associated with the target disease. Upon receiving the user instruction, the client device 10 may place, on the screen, objects showing information on the test associated with the target disease (e.g., questions included in the test, or a link for accessing a website where the test can be taken) or may transition to a screen showing such information.

After step S113, the client device 10 may terminate the sign evaluation process (FIG. 7).

### (5) Training Dataset

The following will describe a training dataset that can be used in supervised learning for constructing a trained model in the present embodiment.

The training dataset includes multiple training data items. The training data items are used for training or evaluating a model to be trained (hereinafter referred to as a “target model”). Each training data item includes a sample ID, input data, and ground truth data.

The sample ID is information that identifies the training data item.

The input data is data that is input to the target model during training or evaluation. The input data corresponds to an example used in the training or evaluation of the target model. As an example, the input data includes data on body movements of a subject performing exercise (i.e., performing the exercise type corresponding to the target model) (i.e., relatively dynamic data) and data on the health state of the subject (i.e., relatively static data). At least part of the data on the subject's body movements is obtained by referring to subject video data (or subject video data and subject depth data) and analyzing the subject's body movements.

The subject video data is data on a subject video that shows the subject performing exercise. The subject video data can be obtained by, for example, capturing the appearance (e.g., whole body) of the subject undergoing an expiratory gas test (e.g., a CPX test) with a camera (e.g., a camera in a smartphone) from the front or from a diagonal front angle (e.g., 45 degrees forward).

The subject depth data is data on the distance (depth) from a depth sensor to each part of the subject performing exercise. The subject depth data can be obtained by operating the depth sensor while capturing the subject video.

Subjects typically include people diagnosed as having a specific disease or a sign of the disease, and people diagnosed as not having the disease or a sign of the disease. The diagnosis is usually conducted by physicians, although physicians' diagnosis may be replaced by determination using an algorithm (which may include a trained model). However, the training data used may be data on a single subject, including data collected during a period when the subject is diagnosed as having a specific disease or a sign of the disease, and data collected during a period when the subject is diagnosed as not having the disease or a sign of the disease. Furthermore, the subjects may include the user to be evaluated for signs of the specific disease during the operation of the information processing system 1.

In the present example, the input data includes at least movement data. The movement data may include elements similar to those in the above-described user movement information (e.g., skeleton information) and can be obtained by referring to the subject video data and analyzing the movements (e.g., skeleton) of the subject performing exercise. The movement data may also be obtained by referring to, in addition to the subject video data, at least one of the following: the subject depth data; or acceleration data measured by a wearable device worn by the subject.

The ground truth data serves as the correct answer to the corresponding input data (example). The target model is trained to provide output closer to the ground truth data in response to the input data (supervised learning). As an example, the ground truth data indicates the presence or absence of the specific disease or a sign of the disease.

The ground truth data represents, for example, the presence or absence of the specific disease or a sign of the disease in the subject who is the origin of the corresponding input data. That is, the ground truth data corresponding to input data obtained from the subject diagnosed as having the specific disease or a sign of the disease has a value indicating the presence of the disease or a sign of the disease. In contrast, the ground truth data corresponding to input data obtained from the subject diagnosed as not having the specific disease or a sign of the disease has a value indicating the absence of the disease or a sign of the disease.

In addition to the movement data alone, at least one of the items listed below may be added to the input data together with the movement data. Alternatively, the input data may include at least one of the items listed below as its element, and a trained model may be constructed that performs inference in response to the input data in a manner similar to the above. In that case, input data during the operation of the trained model will similarly include such an element resulting from sensing the user.
facial expression data (an example of “facial expression information”)
gaze data (an example of “gaze information”)
tremor data (an example of “tremor information”)
assistive device data (an example of “assistive device information”)
voice data (an example of “voice information”)
health state data (an example of “health state information”)

The facial expression data is data (e.g., feature values) on the facial expressions of the subject performing exercise. The facial expression data can be analyzed by applying an algorithm or a trained model to the subject video data. Alternatively, the facial expression data for the training dataset can be obtained by, for example, causing a human viewer to label the subject video.

The gaze data is data (e.g., feature values) on the gaze of the subject performing exercise. The gaze data can be analyzed by applying an algorithm or a trained model to the subject video data.

The tremor data is data (e.g., feature values) on the tremors of the subject performing exercise. The tremor data can be analyzed by applying an algorithm or a trained model to the subject video data. Alternatively, the tremor data for the training dataset can be obtained by, for example, causing a human viewer to label the subject video.

The assistive device data is data (e.g., feature values) on the use of an assistive device (e.g., a walking aid such as a cane, or any other tool used in exercise) by the subject performing exercise. The assistive device data can be analyzed by applying an algorithm or a trained model to the subject video data. Alternatively, the assistive device data for the training dataset can be obtained by, for example, causing a human viewer to label the subject video.

The voice data is data (e.g., feature values) on the voice of the subject performing exercise. The voice data can be analyzed by applying an algorithm or a trained model to sound data collected by a microphone disposed near the subject. Alternatively, the voice data for the training dataset can be obtained by, for example, causing a human listener to label the subject's voice.

The health state data is data on the subject's health state. The health state data can be obtained through various methods. The subject's health state data may be obtained at any time: before, during, or after the subject's exercise. The subject's health state data may be obtained on the basis of a report from the subject or the subject's attending physician, or by extracting information associated with the subject in a medical information system, or through the subject's app (e.g., healthcare app).

The health state includes at least one of the following:
age
sex
height
weight
body fat percentage
muscle mass
bone density
history of present illness
past medical history
medication history
surgical history
lifestyle history (e.g., smoking history, drinking history, activities of daily living (ADL), frailty score, etc.)
family history
results of pulmonary function tests
results of tests other than pulmonary function tests (e.g., blood tests, urine tests, electrocardiography tests (including Holter monitoring tests), cardiac ultrasound tests, X-ray tests, CT tests (including cardiac morphology CT and coronary CT tests), MRI tests, nuclear medicine tests, PET tests, etc.)
data obtained during cardiac rehabilitation (including the Borg index)

Note that a trained model may be constructed for each of multiple health state categories on the basis of (at least part of) the subject's health state. In that case, (at least part of) the user's health state may be referred to for selecting a trained model. In this variation, the input data for the trained model may be either data not based on the user's health state or data based on the user's health state and the user video.

### (6) Interim Summary

As described above, the server 30 in the present embodiment acquires user movement information on a user's movement resulting from analyzing an image in which the user is captured performing exercise of a first type, which is an exercise type selected from multiple exercise types by the user, a related party, or an algorithm. The server 30 evaluates the user for a sign of a target disease on the basis of at least the user movement information and a feature (in the present embodiment, a trained model) related to a symptom of the target disease; the target disease is at least one of a cranial nerve disease or a peripheral nerve/muscle disease, associated with the first type. This reduces the burden on the user and thus facilitates routine evaluation for the sign of the target disease, contributing to early detection of the target disease.

The server 30 may evaluate the user for the sign of the target disease by applying a trained model corresponding to the first type to input data that is based on the user movement information. This enables obtaining a statistically valid evaluation result without the need to create an evaluation algorithm. Furthermore, the use of the trained model corresponding to the first type prevents the evaluation from being affected by differences dependent on the exercise type, such as differences in the postures or movements of the skeleton or other feature points. This can improve the accuracy of the evaluation.

The server 30 may evaluate the user for the sign of the target disease by applying a trained model corresponding to the first type and the target disease to input data that is based on the user movement information. The use of the trained model corresponding to the combination of the first type and the target disease prevents the evaluation from being affected by differences dependent on at least one of the exercise type or the disease, such as differences in the postures or movements of the skeleton or other feature points. This can improve the accuracy of the evaluation.

The user movement information may indicate at least one of the position, velocity, or acceleration of a joint of the user. This enables quantitative representation of the postures or movements of the user's skeleton, allowing for objective evaluation.

The server 30 may acquire at least one of facial expression information on the user's facial expression, gaze information on the user's gaze, tremor information on the user's limb tremor, or assistive device information on the user's use of a walking aid, resulting from analyzing the above image. The server 30 may evaluate the user for the sign of the target disease further on the basis of at least one of the facial expression information, the gaze information, the tremor information, and the assistive device information. This enables evaluation from perspectives other than movements, facilitating the prevention of overlooked signs.

The server 30 may acquire voice information on the voice of the user performing exercise of the first type and evaluate the user for the sign of the target disease further on the basis of the voice information. This enables evaluation from the perspectives of movements and voice, facilitating the prevention of overlooked signs.

In a case where the result of evaluating the user satisfies a predetermined condition, the server 30 may select, from the multiple exercise types, a second type associated with the target disease and different from the first type, and present information on the second type to the user. The server 30 may then perform the sign evaluation for the second type in a similar manner. This can yield a more reliable evaluation result.

In a case where the result of evaluating the user satisfies a predetermined condition, the server 30 may present, to the user, information on a medical institution associated with the target disease. This can prompt the user to consult a medical institution that is strong in treating the target disease and thus to achieve early detection of the target disease.

In a case where the result of evaluating the user satisfies a predetermined condition, the server 30 may present, to the user, information on a test associated with the target disease. This can prompt the user to take a test established for the target disease and thus to achieve early detection of the disease.

### (7) Variations

Variations of the present embodiment will be described.

### (7-1) Variation 1

Variation 1 will be described. Variation 1 is an example of performing the sign evaluation using patterns instead of trained models.

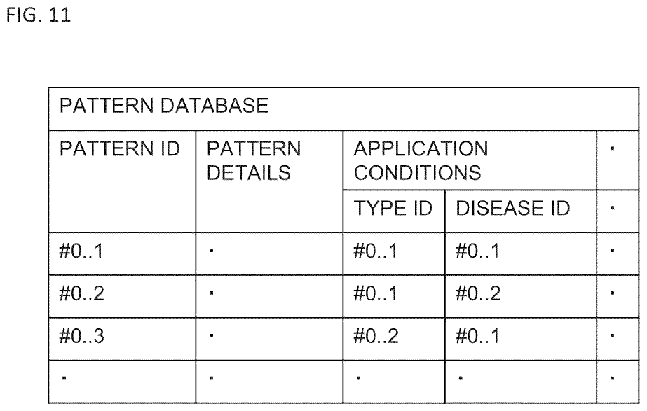

In Variation 1, a pattern database is used in addition to or instead of the model database (FIG. 6). FIG. 11 is a diagram illustrating the data structure of the pattern database in Variation 1.

The pattern database stores pattern information. The pattern information is information on patterns of movement information (e.g., skeleton information) that serve as references for performing evaluation for signs of one or more diseases. The patterns can be determined by, for example, analyzing features of skeletons or other feature points observed while patients with specific diseases or people exhibiting signs of the diseases perform exercise types.

As shown in FIG. 11, the pattern database includes a “pattern ID” field, a “pattern details” field, and an “application conditions” field. The fields are associated with each other.

The “pattern ID” field stores pattern IDs. Each pattern ID is information that identifies the pattern corresponding to the relevant record.

The “pattern details” field stores pattern details information. Each item of the pattern details information is information on the details of the pattern corresponding to the relevant record. The details information includes information capable of identifying the pattern (e.g., information defining conditions related to a specific joint, the orientation, velocity, or acceleration of a specific joint, or time-dependent changes thereof, or information indicating the location where such information is stored).

The “application conditions” field stores application condition information. Each item of the application condition information is information on a condition under which the pattern corresponding to the relevant record is applicable. As an example, the applicable condition information may include information capable of identifying an exercise type (e.g., the above-described type ID) and information capable of identifying a disease (i.e., a target disease). As the information capable of identifying a disease, a disease ID may be defined that uniquely identifies a single disease, or a disease group including multiple diseases.

As a first example of the sign evaluation (step S130 in FIG. 7), the server 30 refers to the pattern database (FIG. 11) to extract records that include, as an application condition, the value of the type ID corresponding to the first type. In this case, the pattern corresponding to each record extracted constitutes a feature related to a symptom of a target disease. The server 30 evaluates the user for a sign of the target disease by comparing the pattern with the user movement information.

A second example of the sign evaluation (S130) is a case where the target disease is specified. The server 30 refers to the pattern database (FIG. 11) to extract records that include, as application conditions, the values of the type ID corresponding to the first type and the disease ID corresponding to the specified target disease. In this case, the pattern corresponding to each record extracted constitutes a feature related to a symptom of the target disease. The server 30 evaluates the user for a sign of the target disease by comparing the pattern with the user movement information. The target disease may be specified by the user, the related party for the user, or an algorithm. Information capable of identifying the specified target disease may be included in the user data, may be transmitted to the server 30 from the related party's terminal, or may be generated by the server 30.

As described above, the server 30 in Variation 1 may evaluate the user for a sign of the target disease by selecting a pattern corresponding to the first type and comparing the pattern with the user movement information. The use of the pattern corresponding to the first type prevents the evaluation from being affected by differences dependent on the exercise type, such as differences in the postures or movements of the skeleton or other feature points. This can improve the accuracy of the evaluation.

The server 30 may evaluate the user for the sign of the target disease by selecting a pattern corresponding to the first type and the target disease and comparing the pattern with the user movement information. The use of the pattern corresponding to the combination of the first type and the target disease prevents the evaluation from being affected by differences dependent on at least one of the exercise type or the disease, such as differences in the postures or movements of the skeleton or other feature points. This can improve the accuracy of the evaluation.

### (8) Other Variations

The storage device 11 may be connected to the client device 10 via the network NW. Each input device or output device may be incorporated in the client device 10. The storage device 31 may be connected to the server 30 via the network NW.

The above description has illustrated an example in which the information processing system 1 in the embodiment is implemented by a client-server system. However, the information processing system 1 in the embodiment may be implemented by a peer-to-peer system or a standalone computer. As an example, the client device 10 may perform the sign evaluation.

Each step in the above-described information processing may be performed by either the client device 10 or the server 30. As an example, instead of the client device 10, the server 30 may analyze the sensing data to acquire the user movement information.

The above description has illustrated an example in which the user video is captured using the camera 16 of the client device 10. However, the user video may be captured using a camera different from the camera 16. The above description has illustrated an example in which the user depth is measured using the depth sensor 17 of the client device 10. However, the user depth may be measured using a depth sensor different from the depth sensor 17.

Acceleration data may be used as part of the input data for the trained model described in the present embodiment or the variations. Alternatively, acceleration data may be referred to for analyzing the user's movements (e.g., skeleton). For example, the acceleration data may be obtained by the acceleration sensor 19, or by an acceleration sensor in a wearable device (not shown) worn by the user, during the recording of the user video.

The information processing system 1 in the present embodiment and the variations is also applicable to a video game in which the progress of the game is controlled depending on the player's body movements. The video game may be a mini-game that can be played during the execution of the above-mentioned therapeutic app, rehabilitation app, or fitness app. As an example, during gameplay, the information processing system 1 performs estimation regarding the user's movements (e.g., skeleton) on the basis of user video. In addition to the user video, the estimation regarding the user's movements may further be performed based on at least one of user depth or user acceleration. On the basis of the result of the estimation regarding the user's movements, the information processing system 1 evaluates how well the user's posture during exercise (e.g., calisthenics) conforms to the ideal posture (model). In accordance with the result of this evaluation (e.g., a numerical value indicating the degree of conformity of the user's posture to the ideal posture), the information processing system 1 may determine one of the items listed below. This can enhance the effect of the video game on promoting the user's health.
the quality (e.g., difficulty level) or quantity of a challenge (e.g., stage, mission, or quest) imposed on the user in the video game
the quality (e.g., type) or quantity of a reward (e.g., in-game currency, item, or bonus) given to the user in the video game
a game parameter related to the progress of the video game (e.g., score or damage)

In addition to or instead of the microphone 18, a microphone of a wearable device (not shown) worn by the user (i.e., a microphone included in or connected to the wearable device) may receive sound waves emitted by the user during the recording of the user video and generate sound data. The sound data may constitute part of the input data for the trained model described in the present embodiment or the variations. The sound emitted by the user is, for example, sound produced by the user's breathing or vocalization.

While an embodiment of the present invention has been described in detail, the scope of the present invention is not limited by the above embodiment. Various improvements and modifications may be made to the above embodiment without departing from the spirit of the present invention. Combinations of the above embodiment and variations are also possible.

1. An information processing apparatus comprising processing circuitry configured to:
acquire type information on a first type that is an exercise type selected from a plurality of exercise types by a user, a related party, or an algorithm;
acquire user movement information on the user's movement resulting from analyzing an image in which the user is captured performing exercise of the first type; and
evaluate the user for a sign of a target disease on the basis of at least the user movement information and a feature related to a symptom of the target disease, the target disease being at least one of a cranial nerve disease or a peripheral nerve/muscle disease, associated with the first type.

2. The information processing apparatus according to claim 1, wherein the processing circuitry is configured to evaluate the user for the sign of the target disease by applying a trained model corresponding to the first type to the user movement information.

3. The information processing apparatus according to claim 2, wherein the processing circuitry is configured to evaluate the user for the sign of the target disease by applying a trained model corresponding to the first type and the target disease to the user movement information.

4. The information processing apparatus according to claim 1, wherein the processing circuitry is configured to evaluate the user for the sign of the target disease by selecting a pattern corresponding to the first type and comparing the pattern with the user movement information.

5. The information processing apparatus according to claim 4, wherein the processing circuitry is configured to evaluate the user for the sign of the target disease by selecting a pattern corresponding to the first type and the target disease and comparing the pattern with the user movement information.

6. The information processing apparatus according to claim 1, wherein the user movement information indicates at least one of a position, a velocity, or an acceleration of a joint of the user.

7. The information processing apparatus according to claim 1, wherein the processing circuitry is configured to:
acquire at least one of facial expression information on the user's facial expression, gaze information on the user's gaze, tremor information on the user's limb tremor, or assistive device information on the user's use of a walking aid, resulting from analyzing the image; and
evaluate the user for the sign of the target disease further on the basis of at least one of the facial expression information, the gaze information, the tremor information, and the assistive device information.

8. The information processing apparatus according to claim 1, wherein the processing circuitry is further configured to:
acquire voice information on voice of the user performing exercise of the first type; and
evaluate the user for the sign of the target disease further on the basis of the voice information.

9. The information processing apparatus according to claim 1, wherein the processing circuitry is further configured to:
select, from the plurality of exercise types, a second type that is an exercise type associated with the target disease and different from the first type in a case where a result of evaluating the user satisfies a predetermined condition; and
present information on the second type to the user.

10. The information processing apparatus according to claim 1, wherein the processing circuitry is configured to present, to the user, information on a medical institution associated with the target disease in a case where a result of evaluating the user satisfies a predetermined condition.

11. The information processing apparatus according to claim 1, wherein the processing circuitry is configured to present, to the user, information on a test associated with the target disease in a case where a result of evaluating the user satisfies a predetermined condition.

12. A method executed by a computer comprising processing circuitry, wherein the processing circuitry executes:
acquiring type information on a first type that is an exercise type selected from a plurality of exercise types by a user, a related party, or an algorithm;
acquiring user movement information on the user's movement resulting from analyzing an image in which the user is captured performing exercise of the first type; and
evaluating the user for a sign of a target disease on the basis of at least the user movement information and a feature related to a symptom of the target disease, the target disease being at least one of a cranial nerve disease or a peripheral nerve/muscle disease, associated with the first type.

13. A system comprising a first information processing apparatus and a second information processing apparatus,
the first information processing apparatus comprising processing circuitry configured to:
acquire type information on a first type that is an exercise type selected from a plurality of exercise types by a user, a related party, or an algorithm;
acquire user movement information on the user's movement resulting from analyzing an image in which the user is captured performing exercise of the first type;
evaluate the user for a sign of a target disease on the basis of at least the user movement information and a feature related to a symptom of the target disease, the target disease being at least one of a cranial nerve disease or a peripheral nerve/muscle disease, associated with the first type; and
transmit, to the second information processing apparatus, information that is based on a result of evaluating the user.
